(19) United States
(12) Patent Application Publication
(10) Pub. No.: US 2022/0323249 A1
(43) Pub. Date: Oct. 13, 2022

(54) Device for preventing drooling

(71) Applicant: Jose Eduardo Lindley, Pembroke Pines, FL (US)

(72) Inventor: Jose Eduardo Lindley, Pembroke Pines, FL (US)

(21) Appl. No.: 17/225,206

(22) Filed: Apr. 8, 2021

(51) Int. Cl. A61F 5/37

(52) U.S. Cl. CPC A61F 5/3707

(57) ABSTRACT

The device is made of two, independent bands. Each band is made of a stretchable material that can be stretched over and around a user's head. The first band is made to be placed around a user's jaw and the top of a user's head. The band comes down and across both sides of the face and around the chin of the user. This first band is used to simultaneously keep the user's jaws, mouth, and lips closed if desired. The second band is made to be placed around user's forehead and is used to keep the first band in place. The second band may or may not be physically attached to the first band.

### BACKGROUND OF THE INVENTION

### Field of the Invention

This invention is directed to a non-therapeutic sleep aid, More particularly, the invention is directed to an anti-drooling harness which may be used by individuals while sleeping or awake.

### Prior Art

Hundreds of devices are registered in the U.S. Patent and Trademark Office as cures for drooling, snoring, or other sleep issues. While the devices are not therapeutic, they do provide a measure of relief in some scenarios. For example, an anti-drooling device is essentially concerned with sanitation and hygiene, but does not cure or treat any disease.

Some are variations on the old idea of sewing a sock that holds a tennis ball on the pajama back to force the user to sleep on his side. Some devices reposition the lower jaw forward; some open nasal air passages; a few others have been designed to prevent the undesirable conditions of drooling, snoring, or talking during sleep. These devices however, tend to suffer from drawbacks. First, they tend to be complicated as the involve rigid components that require elaborate arrangements to secure in position on the user while sleeping. Also, they tend to be too bulky to use in any setting other than sleeping.

In summary, while prior art devices can be partially effective, they suffer from drawbacks.

### BRIEF SUMMARY OF THE INVENTION

The purpose of this invention is to reduce drooling by an individual.The inventive device requires only two interacting straps. A first strap is used to urge the user's mouth into a closed or nearly closed position. A second strap is used to fix the position of the first strap. The interacting straps cooperate to apply a gentle urging to bias and maintain the user's jaw in an anti-drooling position while still allowing the user to open the mouth easily if necessary.

The packaging for the straps may be hygienically sealed, and the seal would be removed before use. Application of the strap should be easy for any adult, even one with some physical impairment. One strap would wrap around the forehead while the second strap would be wrapped from under the chin to over the head of the user to assist in securing the user's upper and lower jawbone and lips together, essentially forcing the mouth gently shut. The fabric is made of a stretchable material so that a user can still force the mouth open to talk and breathe if desired.

The strap would be applied when settling down for sleep and removed when arising from sleep.

### BRIEF DESCRIPTION OF THE SEVERAL VIEWS OF THE DRAWINGS

FIG. 1 shows a perspective view of the device in use.

FIG. 2 shows a perspective view of the device in use.

### DETAILED DESCRIPTION OF THE INVENTION

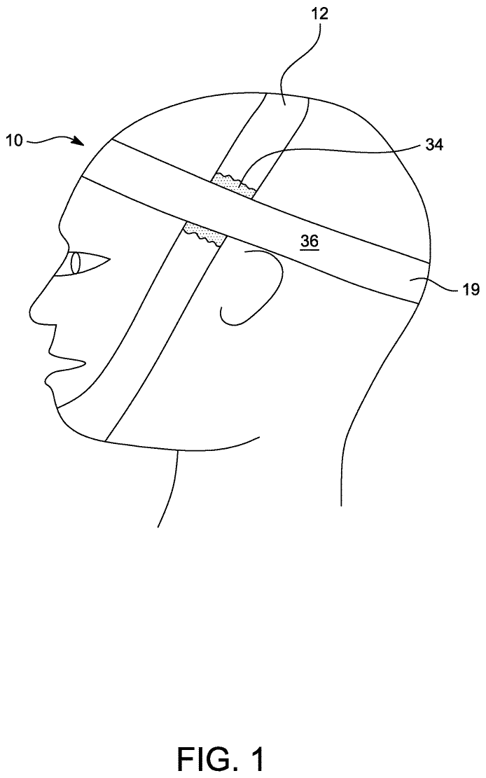

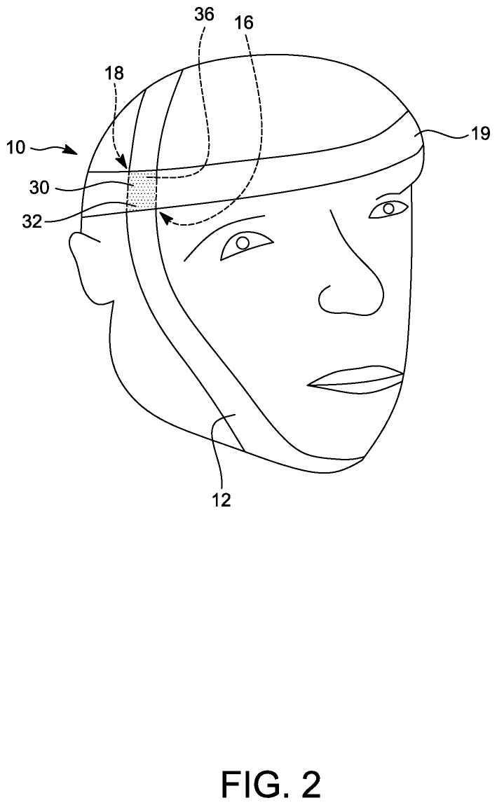

Referring now to FIGS. 1 and 2, the present invention, generally indicated by the numeral 10, is shown. The device is made of two, independent bands. Each band is made of a stretchable material that can be stretched over and around a user's head. The first band 12 is made to be placed around a user's jaw and the top of a user's head. The band comes down and across both sides of the face and around the chin of the user. This first band 12 is used to simultaneously keep the user's jaws, mouth, and lips closed if desired.

It can be appreciated that drooling can be greatly reduced by keeping the user's mouth closed while sleeping. Any such device, to be practical, must be sufficiently comfortable to avoid interfering the user's sleep. Also, the device must be able to stay in place while the user is asleep. Finally, the device 10 should be unobtrusive so that it can be worn when awake so it can be used by those with, e.g., motor control issues that promote drooling.

The invention may be marketed as a one size fits all device as it is flexible. However, the size of the user's head is related to the age of the user. As the device 10 cannot be safely used with infants or toddlers, the device 10 preferably comes in an at least an adult size and youth size. Alternatively, straps 12 or 14 may be lengthwise adjustable so that a desired and comfortable amount of tension to bias the jaw closed can be achieved.

The user's chin may optionally be rested in the middle of the first band 12 to provide additional support. This may also provide support to prevent unintentional removal during sleep.

The second band 14 is made to be placed around user's forehead and is used to keep the first band in place. The second band 14 may or may not be physically attached to the first band. Traction enhancing material 16 such as rubber may be positioned on the interior 18 side of the second band 14, this serving to prevent relative slippage of the bands. Also, hook and loop type fastening material 30 may be placed on on the overlapping area 32 of the respective bands 12, 14 to further enhance stability, the material 34 for the band 12 positioned on an exterior surface 36. This arrangement thus ensures that the device 10 will stay in the desired position once adjusted.

The second band 14 serves to keep the first band in place and prevents it from sliding down either to the back or front of the user's head. By keeping the first band 12 in the correct position, this allows the first band to keep the desired tension necessary to help the jaw stay closed while sleeping. The user may adjust the angle and relative position of the straps 12, 14 to acquire a level of tension that is comfortable.

Since the device is made of stretchable material, this allows the user to talk or breathe through the mouth without removing the device.

FIGS. 1 and 2 show the first band, 12, and second band, 14, around a user's head.

1. A device positioned on a user's head to urge a user's jaw into the closed position to prevent drooling, the device comprising:
a first band formed of an elastic material and sized to encircle a user's head and bias the jaw in a closed position;
a second band sized to encircle a user's forehead;
whereby said first band urges the jaw into the closed position and the second band secures the first band into position.

2. The device of claim 1 wherein the first and second band are made of a stretchable material.

3. The device of claim 1 wherein the first and second band are joined together.

4. The device of claim 1 wherein the first and second band are not joined together.

5. The device of claim wherein the first and second bands have traction enhancing material positioned in an overlapping area.
